(19) United States
(12) Patent Application Publication
(10) Pub. No.: US 2008/0175882 A1
(43) Pub. Date: Jul. 24, 2008

(54) Polymers of aliphatic thioester

(76) Inventors: Mikael O. Trollsas, San Jose, CA (US); Nam D. Pham, San Jose, CA (US); Michael H. Ngo, San Jose, CA (US)

Correspondence Address: SQUIRE, SANDERS & DEMPSEY LLP, 1 MARITIME PLAZA, SUITE 300, SAN FRANCISCO, CA, 94111, US

(21) Appl. No.: 11/657,433

(22) Filed: Jan. 23, 2007

(51) Int. Cl. A61K 31/4745; A61F 2/02

(52) U.S. Cl. 424/423; 514/291; 525/440.05

(57) ABSTRACT

A coating comprising a polymer that comprises units derived from an aliphatic thioester on a medical device is provided.

### FIELD OF THE INVENTION

This invention generally relates polythioester polymers, which is a biomaterial that can be used in biomedical applications such as coating a stent.

### DESCRIPTION OF THE BACKGROUND

Blood vessel occlusions are commonly treated by mechanically enhancing blood flow in the affected vessels, such as by employing a stent. Stents are used not only for mechanical intervention but also as vehicles for providing biological therapy. To effect a controlled delivery of an active agent in stent medication, the stent can be coated with a biocompatible polymeric coating. The biocompatible polymeric coating can function either as a permeable layer or a carrier to allow a controlled delivery of the agent.

Although stents work well mechanically, the chronic issues of restenosis and, to a lesser extent, stent thrombosis remain. Pharmacological therapy in the form of a drug delivery stent appears to be a feasible means to tackle these issues. Polymeric coatings placed onto the stent serve to act both as the drug reservoir and to control the release of the drug. One of the commercially available polymer-coated products is a stent manufactured by Boston Scientific. For example, U.S. Pat. Nos. 5,869,127; 6,099,563; 6,179,817; and 6,197,051, assigned to Boston Scientific Corporation, describe various compositions for coating medical devices. These compositions provide to stents described therein an enhanced biocompatibility and may optionally include a bioactive agent. U.S. Pat. No. 6,231,590 to Scimed Life Systems, Inc., describes a coating composition, which includes a bioactive agent, a collagenous material, or a collagenous coating optionally containing or coated with other bioactive agents.

The nature of the coating polymers plays an important role in defining the surface properties of a coating. For example, coating integrity depends largely on the nature of the polymer forming the coating. For example, a very low T_{g}, amorphous coating material can have unacceptable rheological behavior upon mechanical perturbation such as crimping, balloon expansion, etc. On the other hand, a high T_{g} or highly crystalline coating material introduces brittle fractures in the high strain areas of the stent pattern.

Therefore, there is a need for polymeric materials that can be tailored to meet need of a coating on a medical device.

The polymer and methods of making the polymer disclosed herein address the above-described problems.

### SUMMARY OF THE INVENTION

Provided herein is a polymer comprising units derived from aliphatic thioester. Thioester polymers are biomaterials that can be used for forming fiber, film, coating, particle, and/or gel in many biomedical applications. In addition, a polymer comprising thioester units can have short hydrolysis half-life time, thus allowing the polymer to have tunable degradation rate. The polymer can be used for drug delivery, allowing control of drug release by controlled erosion of the polymer.

In some embodiments, the polymer described herein can be used to form a coating on an implantable device, which can optionally include a bioactive agent. The bioactive agent can be any diagnostic agent, therapeutic agent, or preventive agent. Some examples of such bioactive agents include, but are not limited to, paclitaxel, docetaxel, estradiol, 17-beta-estradiol, nitric oxide donors, super oxide dismutases, super oxide dismutase mimics, 4-amino-2,2,6,6-tetramethylpiperidine-1-oxyl (4-amino-TEMPO), tacrolimus, dexamethasone, rapamycin, rapamycin derivatives, 40-O-(2-hydroxy)ethyl-rapamycin (everolimus), 40-O-(3-hydroxy)propyl-rapamycin, 40-O-[2-(2-hydroxy)ethoxy]ethyl-rapamycin, and 40-O-tetrazole-rapamycin, 40-epi-(N1-tetrazolyl)-rapamycin (ABT-578), γ-hiridun, clobetasol, mometasone, pimecrolimus, imatinib mesylate, or midostaurin, or prodrugs, co-drugs, or combinations of these. In some embodiments, the hydrophilic bioactive agent can be a peptide (e.g., RGD, cRGD or mimetics thereof) or a drug carrying a charge.

A medical device having a coating that includes a thioester polymer described herein can be used to treat, prevent, or ameliorate a medical condition such as atherosclerosis, thrombosis, restenosis, hemorrhage, vascular dissection or perforation, vascular aneurysm, vulnerable plaque, chronic total occlusion, claudication, anastomotic proliferation (for vein and artificial grafts), bile duct obstruction, urethra obstruction, tumor obstruction, and combinations thereof.

### DETAILED DESCRIPTION

Provided herein is a polymer comprising units derived from aliphatic thioester. The thioester polymers can form compositions that are biomaterials that can be used for forming fiber, film, coating, particle, and/or gel in many biomedical applications. In addition, a polymer comprising thioester units can have short hydrolysis half-life time, thus allowing the polymer to have tunable degradation rate. The polymer can be used for drug delivery, allowing control of drug release by controlled erosion of the polymer.

In some embodiments, the polymer described herein can be used to form a coating on an implantable device, which can optionally include a bioactive agent. The bioactive agent can be any diagnostic agent, therapeutic agent, or preventive agent. Some examples of such bioactive agents include, but are not limited to, paclitaxel, docetaxel, estradiol, 17-beta-estradiol, nitric oxide donors, super oxide dismutases, super oxide dismutase mimics, 4-amino-2,2,6,6-tetramethylpiperidine-1-oxyl (4-amino-TEMPO), tacrolimus, dexamethasone, rapamycin, rapamycin derivatives, 40-O-(2-hydroxy)ethyl-rapamycin (everolimus), 40-O-(3-hydroxy)propyl-rapamycin, 40-O-[2-(2-hydroxy)ethoxy]ethyl-rapamycin, and 40-O-tetrazole-rapamycin, 40-epi-(N1-tetrazolyl)-rapamycin (ABT-578), γ-hiridun, clobetasol, mometasone, pimecrolimus, imatinib mesylate, or midostaurin, or prodrugs, co-drugs, or combinations of these. In some embodiments, the hydrophilic bioactive agent can be a peptide (e.g., RGD, cRGD or mimetics thereof) or a drug carrying a charge.

A medical device having a coating that includes a thioester polymer described herein can be used to treat, prevent, or ameliorate a medical condition such as atherosclerosis, thrombosis, restenosis, hemorrhage, vascular dissection or perforation, vascular aneurysm, vulnerable plaque, chronic total occlusion, claudication, anastomotic proliferation (for vein and artificial grafts), bile duct obstruction, urethra obstruction, tumor obstruction, and combinations thereof.

### Aliphatic Thioester

An aliphatic thioester can form a polymer alone or with other monomer(s) to form a homopolymer or a copolymer. A copolymer comprising units derived from an aliphatic thioester can be a block or random copolymer.

In some embodiments, the polymer described herein is a homopolymer. An example of the polymer has a general formula of Formula I:

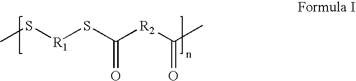

In formula I:

R₁ originates from an aliphatic dithiol. The aliphatic dithiol can be straight chained or branched. In some embodiments, the aliphatic dithiol can be a cyclic aliphatic dithiol, including a cyclic alkyl group(s). In some embodiments, the aliphatic dithiol can include electronic unsaturation including, e.g., C═C bond(s). In some embodiments, the aliphatic dithiol can include arylalkyl groups. In some embodiments, R₁ has a general formula of Formula II —(CH₂)ₘ— where m is a positive integer ranging from 1 to 100, e.g., 2, 3, 4, 5, 6, 7, 8, 9, 10, 11, 12, 13, 14, 15, 16, 17, 18, 19, or 20. In some embodiments, R can include a heteroatom(s) such as oxygen, halo atom(s) (F, Cl, Br or I), S, and N.

R₂ can originate from any organic diacid. For example, R₂ can be an aliphatic diacid. The aliphatic diacid can be straight chained or branched. In some embodiments, the aliphatic diacid can be a cyclic aliphatic diacid, including a cyclic alkyl group(s). In some embodiments, the aliphatic diacid can include electronic unsaturation including, e.g., C═C bond(s). In some embodiments, the aliphatic diacid can include arylalkyl groups. In some embodiments, R₁ has a general formula of Formula II —(CH₂)ₘ— where m is a positive integer ranging from 1 to 100, e.g., 2, 3, 4, 5, 6, 7, 8, 9, 10, 11, 12, 13, 14, 15, 16, 17, 18, 19, or 20. In some embodiments, R₂ can include a heteroatom(s) such as oxygen, halo atom(s) (F, Cl, Br or I), S, and N. In some embodiments, R₂ can include aromatic groups such as phenyl or naphthyl groups. The aromatic groups can further include substituents such as alkyl, halo atom(s) (F, Cl, Br or I), carboxyl, hydroxyl, phosphoryl, sulfonyl, carbonyl, amino, amide, ether, nitro, azo, or combinations thereof.

n is a positive integer ranging from 1 to about 10,000, e.g., from about 10 to about 10,000, from about 20 to about 10,000, from about 50 to about 10,000, from about 100 to about 10,000, from about 500 to about 10,000, or from about 1000 to about 10,000. In some embodiments, n can be an integer from about 10 to about 1000, from about 20 to about 1000, from about 50 to about 1000, or from about 100 to about 1000. Some exemplary values for n are about 10, about 30, about 50, about 60, about 70, about 80, about 90, about 200, about 300, about 400, about 600, about 700, about 800, about 900, about 1500, about 2000, about 3000, about 4000, about 5000, about 6000, about 7000, about 8000, or about 9000.

An example of the polymer of Formula I is shown below:

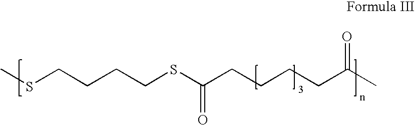

where n is as defined above.

Some other examples of the polymer of Formula I is shown below (Formulae IIIA-IIIF):

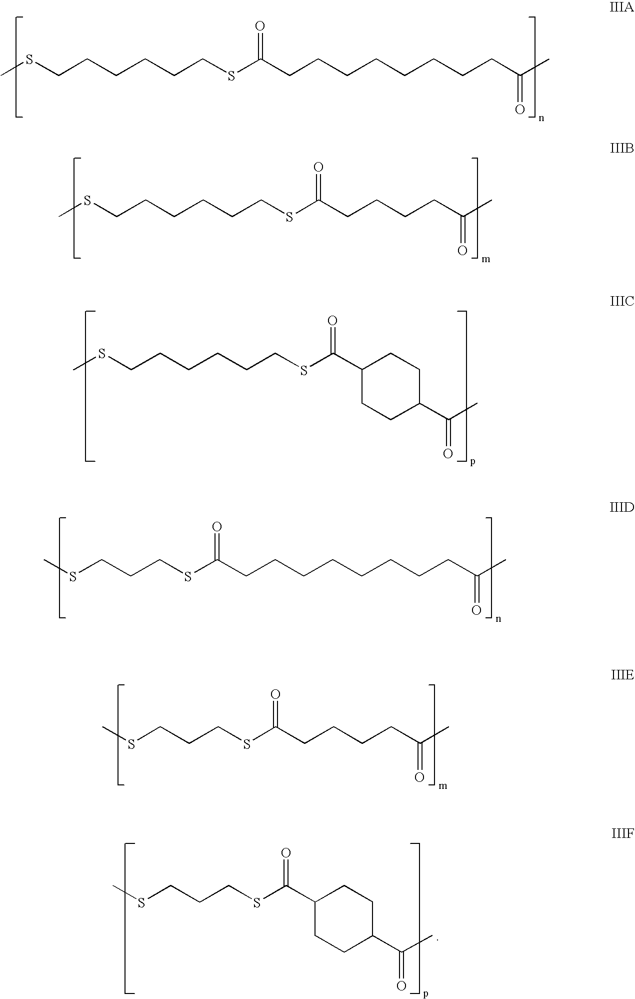

In Formulae IIIA-IIIF, n, m and p are positive integers. The value of n is described above. The values of m and p independently range from 1 to about 10,000, e.g., from about 10 to about 10,000, from about 20 to about 10,000, from about 50 to about 10,000, from about 100 to about 10,000, from about 500 to about 10,000, or from about 1000 to about 10,000. In some embodiments, m and p can be independently integers from about 10 to about 1000, from about 20 to about 1000, from about 50 to about 1000, or from about 100 to about 1000. Some exemplary values for m and p are about 10, about 30, about 50, about 60, about 70, about 80, about 90, about 200, about 300, about 400, about 600, about 700, about 800, about 900, about 1500, about 2000, about 3000, about 4000, about 5000, about 6000, about 7000, about 8000, or about 9000.

The polymer of Formula I can be readily prepared by polymerization of an aliphatic di-thiol and an activated di-ester. An example of the preparation of the polymer of Formula I is shown in Scheme I below, where a di-thiol is allowed to polymerize in dimethyl formamide (DMF) with a nitrophenolate of C10 diacid in the presence a base, triethyl amine.

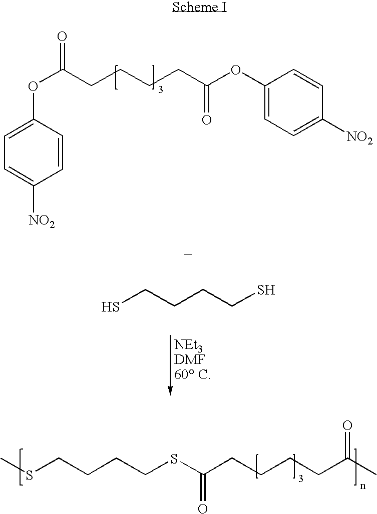

In Scheme I, n is a positive integer as defined above. The activated di-ester can be any activated di-ester. Some examples of the activated di-ester include, but are not limited, nitrophenolate, an N-hydroxysuccinimide di-ester (NHS-di-ester) or an acyl halide.

### Aliphatic Thioester Copolymer

In some embodiments, the polymer described herein is a copolymer including units derived from an aliphatic thioester. For example, the polymer can include units derived from a diol and/or a diamine. The copolymer can be a random copolymer or block copolymer. The random copolymer can be formed by reaction of an activated ester with an aliphatic di-thiol and at least one more monomer selected from a diol, a diamine, or both. For example, a di-thiol, a diol, and/or a diamine can be allowed to polymerize in dimethyl formamide (DMF) with a nitrophenolate of a diacid in the presence a base, triethyl amine, forming a random copolymer having polythioester repeating units, polyester repeating units, polyamide repeating units, poly(ester-amide) repeating units, or poly(thioester-amide) repeating units. The random copolymer can be formed using different molar ratios of dithiol, diol, or diamine monomers. For example, the di-thiol can have a molar ratio (rₙ) ranging from about 0.01 to about 0.99, the diol can have a molar ratio (rₘ) ranging from about 0 to about 0.99, the diamine monomer can have a molar ratio (rₖ) ranging from about 0 to about 0.99, and rₙ+rₘ+rₖ=1.

The di-thiol monomer generally has a formula of HS—R₁—SH (Formula III) where R₁ is an aliphatic group. The aliphatic group can be straight chained or branched. In some embodiments, the aliphatic group can be a cyclic aliphatic group, including a cyclic alkyl group(s). In some embodiments, the aliphatic group can include electronic unsaturation including, e.g., C═C bond(s). In some embodiments, the aliphatic group can include arylalkyl groups. In some embodiments, R₁ has a general formula of Formula II —(CH₂)ₘ— where m is a positive integer ranging from 1 to 100, e.g., 2, 3, 4, 5, 6, 7, 8, 9, 10, 11, 12, 13, 14, 15, 16, 17, 18, 19, or 20. In some embodiments, R₁ can include a heteroatom(s) such as oxygen, halo atom(s) (F, Cl, Br or I), S, and NH.

In some embodiments, the random copolymer can include units having a structure of Formula I, Formula III, or Formulae IIIA, IIIB, IIIC, IIID, IIIE, IIIF, as defined above.

In some embodiments, the thioester polymer described herein is a block copolymer. The block thioester copolymer can be formed by, e.g., coupling polythioester block with other polymeric block(s), e.g., a polyester or poly(ester amide) (PEA) polymer. Representative biocompatible polymers include, but are not limited to, poly(ester amide), polyhydroxyalkanoates (PHA), poly(3-hydroxyalkanoates) such as poly(3-hydroxypropanoate), poly(3-hydroxybutyrate), poly(3-hydroxyvalerate), poly(3-hydroxyhexanoate), poly(3-hydroxyheptanoate) and poly(3-hydroxyoctanoate), poly(4-hydroxyalkanaote) such as poly(4-hydroxybutyrate), poly(4-hydroxyvalerate), poly(4-hydroxyhexanote), poly(4-hydroxyheptanoate), poly(4-hydroxyoctanoate) and copolymers including any of the 3-hydroxyalkanoate or 4-hydroxyalkanoate monomers described herein or blends thereof, poly(D,L-lactide), poly(L-lactide), polyglycolide, poly(D,L-lactide-co-glycolide), poly(L-lactide-co-glycolide), polycaprolactone, poly(lactide-co-caprolactone), poly(glycolide-co-caprolactone), poly(dioxanone), poly(ortho esters), poly(trimethylene carbonate), poly(anhydrides), poly(tyrosine carbonates) and derivatives thereof, poly(tyrosine ester) and derivatives thereof, poly(imino carbonates), poly(glycolic acid-co-trimethylene carbonate), polyphosphoester, polyphosphoester urethane, poly(amino acids), polycyanoacrylates, poly(iminocarbonate), polyurethanes, polyphosphazenes, silicones, polyesters, polyolefins, polyisobutylene and ethylene-alphaolefin copolymers, acrylic polymers and copolymers, vinyl halide polymers and copolymers, such as polyvinyl chloride, polyvinyl ethers, such as polyvinyl methyl ether, polyvinylidene halides, such as polyvinylidene chloride, polyacrylonitrile, polyvinyl ketones, polyvinyl aromatics, such as polystyrene, polyvinyl esters, such as polyvinyl acetate, copolymers of vinyl monomers with each other and olefins, such as ethylene-methyl methacrylate copolymers, acrylonitrile-styrene copolymers, ABS resins, and ethylene-vinyl acetate copolymers, polyamides, such as Nylon 66 and polycaprolactam, alkyd resins, polycarbonates, polyoxymethylenes, polyimides, polyethers, poly(glyceryl sebacate), poly(propylene fumarate), poly(n-butyl methacrylate), poly(sec-butyl methacrylate), poly(isobutyl methacrylate), poly(tert-butyl methacrylate), poly(n-propyl methacrylate), poly(isopropyl methacrylate), poly(ethyl methacrylate), poly(methyl methacrylate), epoxy resins, polyurethanes, rayon, rayon-triacetate, cellulose acetate, cellulose butyrate, cellulose acetate butyrate, cellophane, cellulose nitrate, cellulose propionate, cellulose ethers, carboxymethyl cellulose, polyethers such as poly(ethylene glycol) (PEG), copoly(ether-esters) (e.g. PEO/PLA), polyalkylene oxides such as poly(ethylene oxide), poly(propylene oxide), poly(ether ester), polyalkylene oxalates, polyphosphazenes, phosphoryl choline, choline, poly(aspirin), polymers and co-polymers of hydroxyl bearing monomers such as HEMA, hydroxypropyl methacrylate (HPMA), hydroxypropylmethacrylamide, PEG acrylate (PEGA), PEG methacrylate, 2-methacryloyloxyethylphosphorylcholine (MPC) and n-vinyl pyrrolidone (VP), carboxylic acid bearing monomers such as methacrylic acid (MA), acrylic acid (AA), alkoxymethacrylate, alkoxyacrylate, and 3-trimethylsilylpropyl methacrylate (TMSPMA), poly(styrene-isoprene-styrene)-PEG (SIS-PEG), polystyrene-PEG, polyisobutylene-PEG, polycaprolactone-PEG (PCL-PEG), PLA-PEG, poly(methyl methacrylate)-PEG (PMMA-PEG), polydimethylsiloxane-co-PEG (PDMS-PEG), poly(vinylidene fluoride)-PEG (PVDF-PEG), PLURONIC™ surfactants (polypropylene oxide-co-polyethylene glycol), poly(tetramethylene glycol), hydroxy functional poly(vinyl pyrrolidone), biomolecules such as collagen, chitosan, alginate, fibrin, fibrinogen, cellulose, starch, collagen, dextran, dextrin, fragments and derivatives of hyaluronic acid, heparin, fragments and derivatives of heparin, glycosamino glycan (GAG), GAG derivatives, polysaccharide, elastin, chitosan, alginate, or combinations thereof. In some embodiments, the block copolymer described herein can exclude any one of the aforementioned polymers.

As used herein, the terms poly(D,L-lactide), poly(L-lactide), poly(D,L-lactide-co-glycolide), and poly(L-lactide-co-glycolide) can be used interchangeably with the terms poly(D,L-lactic acid), poly(L-lactic acid), poly(D,L-lactic acid-co-glycolic acid), or poly(L-lactic acid-co-glycolic acid), respectively.

In some embodiments, the random copolymer can include units having a structure of Formula I or Formula III, as defined above.

In some embodiments, the block copolymer described herein can include at least one poly(ester amide) block.

Biocompatible polymers useable for forming a block copolymer with poly(aliphatic thioester) are described below. Methods of forming a block copolymer are well documented in the art. Some examples of forming a block copolymer by coupling polymeric blocks are described in Preparative Methods of Polymer Chemistry, 3^{rd} Edition, Wayne Sorenson, Fred Sweeny, Tod W. Campbell, Eds., John Wiley, 2001. Some other examples of forming the copolymer described herein are described in, for example, D. Braun, et al., Polymer Synthesis: Theory and Practice. Fundamentals, Methods, Experiments. 3^{rd} Ed., Springer, 2001; Hans R. Kricheldorf, Handbook of Polymer Synthesis, Marcel Dekker Inc., 1992; G. Odian, Principles of Polymerization, 3^{rd} ed. John Wiley & Sons, 1991).

### Other Polymers

A coating can be formed of the aliphatic thioester polymer described herein alone or with one or more other polymers. Representative polymers include, but are not limited to, poly(ester amide), polyhydroxyalkanoates (PHA), poly(3-hydroxyalkanoates) such as poly(3-hydroxypropanoate), poly(3-hydroxybutyrate), poly(3-hydroxyvalerate), poly(3-hydroxyhexanoate), poly(3-hydroxyheptanoate) and poly(3-hydroxyoctanoate), poly(4-hydroxyalkanaote) such as poly(4-hydroxybutyrate), poly(4-hydroxyvalerate), poly(4-hydroxyhexanote), poly(4-hydroxyheptanoate), poly(4-hydroxyoctanoate) and copolymers including any of the 3-hydroxyalkanoate or 4-hydroxyalkanoate monomers described herein or blends thereof, poly(D,L-lactide), poly(L-lactide), polyglycolide, poly(D,L-lactide-co-glycolide), poly(L-lactide-co-glycolide), polycaprolactone, poly(lactide-co-caprolactone), poly(glycolide-co-caprolactone), poly(dioxanone), poly(ortho esters), poly(trimethylene carbonate), poly(anhydrides), poly(tyrosine carbonates) and derivatives thereof, poly(tyrosine ester) and derivatives thereof, poly(imino carbonates), poly(glycolic acid-co-trimethylene carbonate), polyphosphoester, polyphosphoester urethane, poly(amino acids), polycyanoacrylates, poly(iminocarbonate), polyurethanes, polyphosphazenes, silicones, polyesters, polyolefins, polyisobutylene and ethylene-alphaolefin copolymers, acrylic polymers and copolymers, vinyl halide polymers and copolymers, such as polyvinyl chloride, polyvinyl ethers, such as polyvinyl methyl ether, polyvinylidene halides, such as polyvinylidene chloride, polyacrylonitrile, polyvinyl ketones, polyvinyl aromatics, such as polystyrene, polyvinyl esters, such as polyvinyl acetate, copolymers of vinyl monomers with each other and olefins, such as ethylene-methyl methacrylate copolymers, acrylonitrile-styrene copolymers, ABS resins, and ethylene-vinyl acetate copolymers, polyamides, such as Nylon 66 and polycaprolactam, alkyd resins, polycarbonates, polyoxymethylenes, polyimides, polyethers, poly(glyceryl sebacate), poly(propylene fumarate), poly(n-butyl methacrylate), poly(sec-butyl methacrylate), poly(isobutyl methacrylate), poly(tert-butyl methacrylate), poly(n-propyl methacrylate), poly(isopropyl methacrylate), poly(ethyl methacrylate), poly(methyl methacrylate), epoxy resins, polyurethanes, rayon, rayon-triacetate, cellulose acetate, cellulose butyrate, cellulose acetate butyrate, cellophane, cellulose nitrate, cellulose propionate, cellulose ethers, carboxymethyl cellulose, polyethers such as poly(ethylene glycol) (PEG), copoly(ether-esters) (e.g. PEO/PLA), polyalkylene oxides such as poly(ethylene oxide), poly(propylene oxide), poly(ether ester), polyalkylene oxalates, polyphosphazenes, phosphoryl choline, choline, poly(aspirin), polymers and co-polymers of hydroxyl bearing monomers such as HEMA, hydroxypropyl methacrylate (HPMA), hydroxypropylmethacrylamide, PEG acrylate (PEGA), PEG methacrylate, 2-methacryloyloxyethylphosphorylcholine (MPC) and n-vinyl pyrrolidone (VP), carboxylic acid bearing monomers such as methacrylic acid (MA), acrylic acid (AA), alkoxymethacrylate, alkoxyacrylate, and 3-trimethylsilylpropyl methacrylate (TMSPMA), poly(styrene-isoprene-styrene)-PEG (SIS-PEG), polystyrene-PEG, polyisobutylene-PEG, polycaprolactone-PEG (PCL-PEG), PLA-PEG, poly(methyl methacrylate)-PEG (PMMA-PEG), polydimethylsiloxane-co-PEG (PDMS-PEG), poly(vinylidene fluoride)-PEG (PVDF-PEG), PLURONIC™ surfactants (polypropylene oxide-co-polyethylene glycol), poly(tetramethylene glycol), hydroxy functional poly(vinyl pyrrolidone), biomolecules such as collagen, chitosan, alginate, fibrin, fibrinogen, cellulose, starch, collagen, dextran, dextrin, fragments and derivatives of hyaluronic acid, heparin, fragments and derivatives of heparin, glycosamino glycan (GAG), GAG derivatives, polysaccharide, elastin, chitosan, alginate, or combinations thereof. In some embodiments, the coating described herein can exclude any one of the aforementioned polymers.

In some embodiments, the coating can further include a biobeneficial material. The biobeneficial material can be polymeric or non-polymeric. The biobeneficial material is preferably substantially non-toxic, non-antigenic and non-immunogenic. A biobeneficial material is one that enhances the biocompatibility of a device by being non-fouling, hemocompatible, actively non-thrombogenic, or anti-inflammatory, all without depending on the release of a pharmaceutically active agent.

Representative biobeneficial materials include, but are not limited to, polyethers such as poly(ethylene glycol), copoly(ether-esters) (e.g. PEO/PLA), polyalkylene oxides such as poly(ethylene oxide), poly(propylene oxide), poly(ether ester), polyalkylene oxalates, polyphosphazenes, phosphoryl choline, choline, poly(aspirin), polymers and co-polymers of hydroxyl bearing monomers such as hydroxyethyl methacrylate (HEMA), hydroxypropyl methacrylate (HPMA), hydroxypropylmethacrylamide, poly(ethylene glycol) acrylate (PEGA), PEG methacrylate, 2-methacryloyloxyethylphosphorylcholine (MPC) and n-vinyl pyrrolidone (VP), carboxylic acid bearing monomers such as methacrylic acid (MA), acrylic acid (AA), alkoxymethacrylate, alkoxyacrylate, and 3-trimethylsilylpropyl methacrylate (TMSPMA), poly(styrene-isoprene-styrene)-PEG (SIS-PEG), polystyrene-PEG, polyisobutylene-PEG, polycaprolactone-PEG (PCL-PEG), PLA-PEG, poly(methyl methacrylate)-PEG (PMMA-PEG), polydimethylsiloxane-co-PEG (PDMS-PEG), poly(vinylidene fluoride)-PEG (PVDF-PEG), PLURONIC™ surfactants (polypropylene oxide-co-polyethylene glycol), poly(tetramethylene glycol), hydroxy functional poly(vinyl pyrrolidone), biomolecules such as fibrin, fibrinogen, cellulose, starch, collagen, dextran, dextrin, hyaluronic acid, fragments and derivatives of hyaluronic acid, heparin, fragments and derivatives of heparin, glycosamino glycan (GAG), GAG derivatives, polysaccharide, elastin, chitosan, alginate, silicones, PolyActive™, or combinations thereof. In some embodiments, the coating can exclude any one of the aforementioned polymers.

The term PolyActive™ refers to a block copolymer having flexible poly(ethylene glycol) and poly(butylene terephthalate) blocks (PEGT/PBT). PolyActive™ is intended to include AB, ABA, BAB copolymers having such segments of PEG and PBT (e.g., poly(ethylene glycol)-block-poly(butyleneterephthalate)-block poly(ethylene glycol) (PEG-PBT-PEG).

In a preferred embodiment, the biobeneficial material can be a polyether such as poly(ethylene glycol) (PEG) or polyalkylene oxide.

### Bioactive Agents

In some embodiments, a coating that includes a aliphatic thioester polymer described herein can optionally include one or more bioactive agents. These bioactive agents can be any agent which is a therapeutic, prophylactic, or diagnostic agent. These agents can have anti-proliferative or anti-inflammatory properties or can have other properties such as antineoplastic, antiplatelet, anti-coagulant, anti-fibrin, antithrombotic, antimitotic, antibiotic, antiallergic, or antioxidant properties. Moreover, these agents can be cystostatic agents, agents that promote the healing of the endothelium, or agents that promote the attachment, migration and proliferation of endothelial cells while quenching smooth muscle cell proliferation. Examples of suitable therapeutic and prophylactic agents include synthetic inorganic and organic compounds, proteins and peptides, polysaccharides and other sugars, lipids, and DNA and RNA nucleic acid sequences having therapeutic, prophylactic or diagnostic activities. Nucleic acid sequences include genes, antisense molecules, which bind to complementary DNA to inhibit transcription, and ribozymes. Some other examples of bioactive agents include antibodies, receptor ligands, enzymes, adhesion peptides, blood clotting factors, inhibitors or clot dissolving agents, such as streptokinase and tissue plasminogen activator, antigens for immunization, hormones and growth factors, oligonucleotides such as antisense oligonucleotides and ribozymes and retroviral vectors for use in gene therapy. Examples of anti-proliferative agents include rapamycin and its functional or structural derivatives, 40-O-(2-hydroxy)ethyl-rapamycin (everolimus), and its functional or structural derivatives, paclitaxel and its functional and structural derivatives. Examples of rapamycin derivatives include ABT-578, 40-O-(3-hydroxy)propyl-rapamycin, 40-O-[2-(2-hydroxy)ethoxy]ethyl-rapamycin, and 40-O-tetrazole-rapamycin. Examples of paclitaxel derivatives include docetaxel. Examples of antineoplastics and/or antimitotics include methotrexate, azathioprine, vincristine, vinblastine, fluorouracil, doxorubicin hydrochloride (e.g. Adriamycin® from Pharmacia & Upjohn, Peapack N.J.), and mitomycin (e.g. Mutamycin® from Bristol-Myers Squibb Co., Stamford, Conn.). Examples of such antiplatelets, anticoagulants, antifibrin, and antithrombins include sodium heparin, low molecular weight heparins, heparinoids, hirudin, argatroban, forskolin, vapiprost, prostacyclin and prostacyclin analogues, dextran, D-phe-pro-arg-chloromethylketone (synthetic antithrombin), dipyridamole, glycoprotein IIb/IIIa platelet membrane receptor antagonist antibody, recombinant hirudin, thrombin inhibitors such as Angiomax (Biogen, Inc., Cambridge, Mass.), calcium channel blockers (such as nifedipine), colchicine, fibroblast growth factor (FGF) antagonists, fish oil (omega 3-fatty acid), histamine antagonists, lovastatin (an inhibitor of HMG-CoA reductase, a cholesterol lowering drug, brand name Mevacor® from Merck & Co., Inc., Whitehouse Station, N.J.), monoclonal antibodies (such as those specific for Platelet-Derived Growth Factor (PDGF) receptors), nitroprusside, phosphodiesterase inhibitors, prostaglandin inhibitors, suramin, serotonin blockers, steroids, thioprotease inhibitors, triazolopyrimidine (a PDGF antagonist), super oxide dismutases, super oxide dismutase mimetic, 4-amino-2,2,6,6-tetramethylpiperidine-1-oxyl (4-amino-TEMPO), estradiol, anticancer agents, dietary supplements such as various vitamins, and a combination thereof. Examples of anti-inflammatory agents including steroidal and non-steroidal anti-inflammatory agents include biolimus, tacrolimus, dexamethasone, clobetasol, corticosteroids or combinations thereof. Examples of such cytostatic substances include angiopeptin, angiotensin converting enzyme inhibitors such as captopril (e.g. Capoten® and Capozide® from Bristol-Myers Squibb Co., Stamford, Conn.), cilazapril or lisinopril (e.g. Prinivil® and Prinzide® from Merck & Co., Inc., Whitehouse Station, N.J.). An example of an antiallergic agent is permirolast potassium. Other therapeutic substances or agents which may be appropriate include alpha-interferon, pimecrolimus, imatinib mesylate, midostaurin, and genetically engineered epithelial cells. The foregoing substances can also be used in the form of prodrugs or co-drugs thereof. The foregoing substances also include metabolites thereof and/or prodrugs of the metabolites. The foregoing substances are listed by way of example and are not meant to be limiting. Other active agents which are currently available or that may be developed in the future are equally applicable.

In some embodiments, a coating including an aliphatic thioester polymer described herein can specifically exclude any one or more of the above described agents.

The dosage or concentration of the bioactive agent required to produce a favorable therapeutic effect should be less than the level at which the bioactive agent produces toxic effects and greater than the level at which non-therapeutic results are obtained. The dosage or concentration of the bioactive agent can depend upon factors such as the particular circumstances of the patient, the nature of the trauma, the nature of the therapy desired, the time over which the ingredient administered resides at the vascular site, and if other active agents are employed, the nature and type of the substance or combination of substances. Therapeutically effective dosages can be determined empirically, for example by infusing vessels from suitable animal model systems and using immunohistochemical, fluorescent or electron microscopy methods to detect the agent and its effects, or by conducting suitable in vitro studies. Standard pharmacological test procedures to determine dosages are understood by those of ordinary skill in the art.

### Examples of Medical Devices

As used herein, a medical device may be any suitable medical substrate that can be implanted in a human or veterinary patient. Examples of such medical devices include self-expandable stents, balloon-expandable stents, stent-grafts, grafts (e.g., aortic grafts), heart valve prostheses, cerebrospinal fluid shunts, pacemaker electrodes, catheters, and endocardial leads (e.g., FINELINE and ENDOTAK, available from Guidant Corporation, Santa Clara, Calif.), anastomotic devices and connectors, orthopedic implants such as screws, spinal implants, and electro-stimulatory devices. The underlying structure of the device can be of virtually any design. The device can be made of a metallic material or an alloy such as, but not limited to, cobalt chromium alloy (ELGILOY), stainless steel (316L), high nitrogen stainless steel, e.g., BIODUR 108, cobalt chrome alloy L-605, “MP35N,” “MP20N,” ELASTINITE (Nitinol), tantalum, nickel-titanium alloy, platinum-iridium alloy, gold, magnesium, or combinations thereof. “MP35N” and “MP20N” are trade names for alloys of cobalt, nickel, chromium and molybdenum available from Standard Press Steel Co., Jenkintown, Pa. “MP35N” consists of 35% cobalt, 35% nickel, 20% chromium, and 10% molybdenum. “MP20N” consists of 50% cobalt, 20% nickel, 20% chromium, and 10% molybdenum. Devices made from bioabsorbable (e.g., bioabsorbable stent) or biostable polymers could also be used with the embodiments of the present invention.

### Method of Use

Preferably, the medical device is a stent. The stent described herein is useful for a variety of medical procedures, including, by way of example, treatment of obstructions caused by tumors in bile ducts, esophagus, trachea/bronchi and other biological passageways. A stent having the above-described coating is particularly useful for treating diseased regions of blood vessels caused by lipid deposition, monocyte or macrophage infiltration, or dysfunctional endothelium or a combination thereof, or occluded regions of blood vessels caused by abnormal or inappropriate migration and proliferation of smooth muscle cells, thrombosis, and restenosis. Stents may be placed in a wide array of blood vessels, both arteries and veins. Representative examples of sites include the iliac, renal, carotid and coronary arteries.

For implantation of a stent, an angiogram is first performed to determine the appropriate positioning for stent therapy. An angiogram is typically accomplished by injecting a radiopaque contrasting agent through a catheter inserted into an artery or vein as an x-ray is taken. A guidewire is then advanced through the lesion or proposed site of treatment. Over the guidewire is passed a delivery catheter that allows a stent in its collapsed configuration to be inserted into the passageway. The delivery catheter is inserted either percutaneously or by surgery into the femoral artery, radial artery, brachial artery, femoral vein, or brachial vein, and advanced into the appropriate blood vessel by steering the catheter through the vascular system under fluoroscopic guidance. A stent having the above-described coating may then be expanded at the desired area of treatment. A post-insertion angiogram may also be utilized to confirm appropriate positioning.

The implantable device can be implanted in any mammal, e.g., an animal or a human being. In some embodiments, the implantable device can be implanted in a patient in need of treatment by the implantable device. The treatment can be angioplasty or other type of treatments involving an implantable device.

A patient who receives the implantable device described herein can be male or female under normal body condition (e.g., normal weight) or abnormal body condition (e.g., underweight or overweight). The patient can be in any age, preferably, the patient is in an age ranging from about 40 to 70 years. An index for measuring the body condition of a patient is BMI (body mass index). A patient can have a BMI ranging from about 18 to about 30 or above.

The implantable device described herein can be used to treat or ameliorate a medical condition such as atherosclerosis, thrombosis, restenosis, hemorrhage, vascular dissection or perforation, vascular aneurysm, vulnerable plaque, chronic total occlusion, claudication, type-II diabetes, anastomotic proliferation for vein and artificial grafts, bile duct obstruction, ureter obstruction, tumor obstruction, or combinations thereof.

### EXAMPLES

The embodiments of the present invention will be illustrated by the following prophetic examples. All parameters and data are not to be construed to unduly limit the scope of the embodiments of the invention.

### Example 1

### Coating a Stent with an Aliphatic Thioester Polymer and Paclitaxel

A first composition can be prepared by mixing the following components:

about 2.0% (w/w) of the aliphatic thioester polymer of formula III;
about 0.2% (w/w) of paclitaxel; and
the balance a 50/50 (w/w) blend of chloroform and 1,1,2-trichloroethane.

The composition can be applied onto the surface of bare 12 mm small VISION™ stent (Guidant Corp.). The coating can be sprayed and dried to form a drug reservoir layer. A spray coater can be used having a 0.014 round nozzle maintained at ambient temperature with a feed pressure 2.5 psi (0.17 atm) and an atomization pressure of about 15 psi (1.02 atm). About 20 μg of the coating can be applied at per one spray pass. About 180 μg of wet coating can be applied, and the stent can be dried for about 10 seconds in a flowing air stream at about 50° C. between the spray passes. The stents can be baked at about 50° C. for about one hour, yielding a drug reservoir layer composed of approximately 150 μg of the aliphatic thioester polymer of formula III and about 14 μg of paclitaxel.

### Example 2

### Coating a Stent with an Aliphatic Thioester Polymer and Everolimus

A first composition can be prepared by mixing the following components:

about 2.0% (w/w) of an aliphatic thioester polymer of formula III;
about 0.2% (w/w) of paclitaxel; and
the balance a 50/50 (w/w) blend of chloroform and 1,1,2-trichloroethane.

The composition can be applied onto the surface of bare 12 mm small VISION™ stent (Guidant Corp.). The coating can be sprayed and dried to form a drug reservoir layer. A spray coater can be used having a 0.014 round nozzle maintained at ambient temperature with a feed pressure 2.5 psi (0.17 atm) and an atomization pressure of about 15 psi (1.02 atm). About 20 μg of the coating can be applied at per one spray pass. About 180 μg of wet coating can be applied, and the stent can be dried for about 10 seconds in a flowing air stream at about 50° C. between the spray passes. The stents can be baked at about 50° C. for about one hour, yielding a drug reservoir layer composed of approximately 150 μg of the aliphatic thioester polymer of formula III and about 14 μg of everolimus.

A second composition can be prepared by mixing the following components:

about 2.0% (w/w) of the aliphatic thioester polymer of formula III;
the balance a 50/50 (w/w) blend of acetone and dimethylformamide.

The second composition can be applied onto the dried drug reservoir layer to form a biobeneficial topcoat layer using the same spraying technique and equipment used for applying the reservoir. About 120 μg of wet coating can be applied followed by drying and baking at about 50° C. for about 2 hours, yielding a dry topcoat layer having solids content of about 100 μg.

While particular embodiments of the present invention have been shown and described, it will be obvious to those skilled in the art that changes and modifications can be made without departing from this invention in its broader aspects. Therefore, the appended claims are to encompass within their scope all such changes and modifications as fall within the true spirit and scope of this invention.

What is claimed is:

1. A coating on a medical device, comprising a polymer comprising aliphatic thioester units derived from an aliphatic di-thiol and a diacid.

2. The coating of claim 1, wherein the polymer is a homopolymer.

3. The coating of claim 1, wherein the polymer is a copolymer.

4. The coating of claim 3, wherein the copolymer is a random or block copolymer.

5. The coating of claim 2, wherein the homopolymer comprises a structure of Formula I: 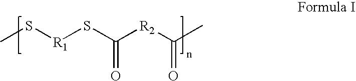
wherein R₁ is an aliphatic group,
wherein R₂ is an organic group, and
wherein n is a positive integer from 1 to about 10,000.

6. The coating of claim 5, wherein the homopolymer comprises a structure of Formula III: 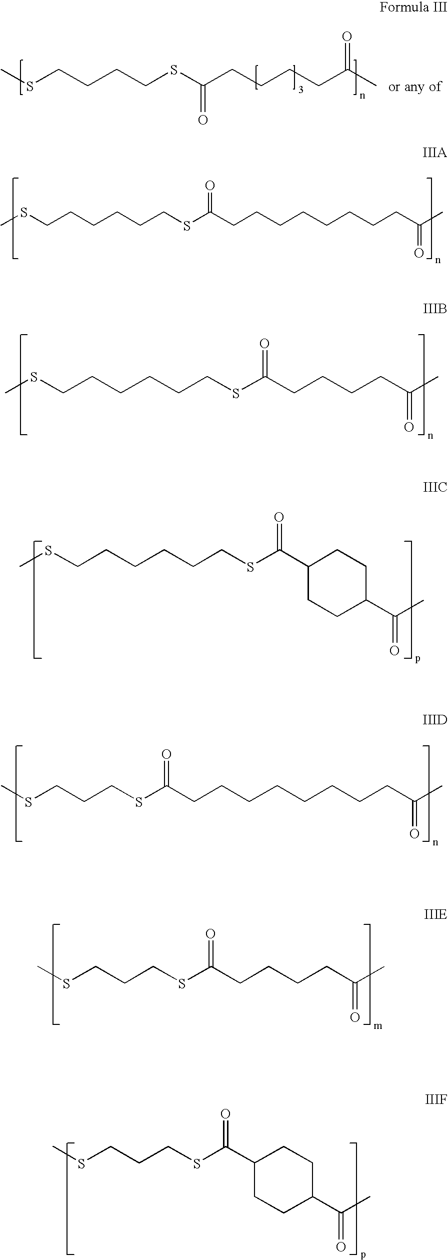
wherein n, m and p are independent positive integers ranging from 1 to about 10,000.

7. The coating of claim 4, wherein the random or block copolymer comprises a structure of Formula I: 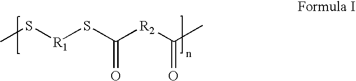
wherein R₁ is an aliphatic group,
wherein R₂ is an organic group, and
wherein n is a positive integer from 1 to about 10,000.

8. The coating of claim 4, wherein the random or block copolymer comprises a structure of Formula III: 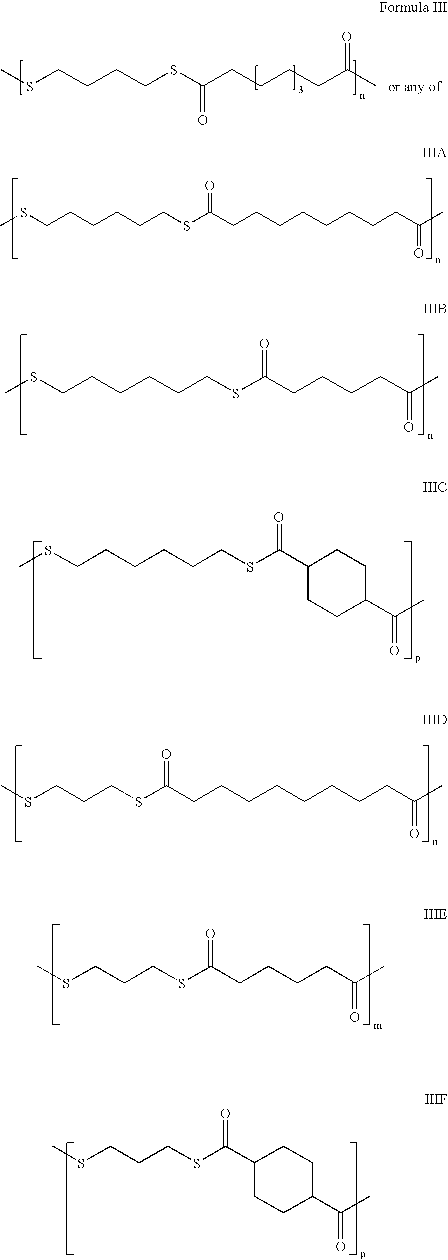
wherein n, m and p are independent positive integers ranging from 1 to about 10,000.

9. The coating of claim 4, wherein the random or block copolymer comprises units derived from a monomer selected from a diol, a diamine, or a combination thereof.

10. The coating of claim 9, wherein the aliphatic di-thiol has a molar ratio (rₙ) from about 0.01 to 0.99,
wherein the diol has a molar ratio (rₘ) from about 0 to about 0.99,
wherein the diamine has a molar ratio (rₖ) from about 0 to about 0.99, and
wherein rₙ+rₘ+rₖ=1.

11. The coating of claim 4, which is a block copolymer, wherein the block copolymer comprises a poly(ester amide) (PEA) block.

12. The coating of claim 1, further comprising at least one other polymer.

13. The coating of claim 12, wherein the at least one other polymer is a poly(ester amide) (PEA) polymer.

14. The coating of claim 1, further comprising a bioactive agent.

15. The coating of claim 1, further comprising a bioactive agent selected from paclitaxel, docetaxel, estradiol, 17-beta-estradiol, nitric oxide donors, super oxide dismutases, super oxide dismutase mimics, 4-amino-2,2,6,6-tetramethylpiperidine-1-oxyl (4-amino-TEMPO), tacrolimus, dexamethasone, rapamycin, rapamycin derivatives, 40-O-(2-hydroxy)ethyl-rapamycin (everolimus), 40-O-(3-hydroxy)propyl-rapamycin, 40-O-[2-(2-hydroxy)ethoxy]ethyl-rapamycin, and 40-O-tetrazole-rapamycin, 40-epi-(N1-tetrazolyl)-rapamycin (ABT-578), γ-hiridun, clobetasol, mometasone, pimecrolimus, imatinib mesylate, or midostaurin, or prodrugs, co-drugs, or combinations of these.

16. The coating of claim 15, wherein the bioactive agent is everolimus.

17. The coating of claim 1, wherein the medical device is a stent.

18. The coating of claim 17, wherein the medical device is a bioabsorbable stent.

19. A medical device comprising a polymer comprising aliphatic thioester units derived from an aliphatic di-thiol and a diacid.

20. The medical device of claim 19, which is a bioabsorbable stent.

21. A method of treating, preventing or ameliorating a medical condition, comprising implanting in a human being a medical device comprising the coating of claim 1, wherein the medical condition is selected from atherosclerosis, thrombosis, restenosis, hemorrhage, vascular dissection or perforation, vascular aneurysm, vulnerable plaque, chronic total occlusion, claudication, anastomotic proliferation for vein and artificial grafts, bile duct obstruction, urethra obstruction, tumor obstruction, diabetic vascular disease, and combinations thereof.

22. A method of treating, preventing or ameliorating a medical condition, comprising implanting in a human being a medical device comprising the coating of claim 15, wherein the medical condition is selected from atherosclerosis, thrombosis, restenosis, hemorrhage, vascular dissection or perforation, vascular aneurysm, vulnerable plaque, chronic total occlusion, claudication, anastomotic proliferation for vein and artificial grafts, bile duct obstruction, urethra obstruction, tumor obstruction, diabetic vascular disease, and combinations thereof.

23. A composition comprising a polymer comprising aliphatic thioester units derived from an aliphatic di-thiol and a diacid.

24. The composition of claim 23, wherein the polymer is a homopolymer.

25. The composition of claim 23, wherein the polymer is a copolymer.

26. The composition of claim 25, wherein the copolymer is a random or block copolymer.

27. The composition of claim 24, wherein the homopolymer comprises a structure of Formula I: 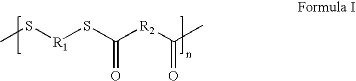
wherein R₁ is an aliphatic group,
wherein R₂ is an organic group, and
wherein n is a positive integer from 1 to about 10,000.

28. The composition of claim 27, wherein the homopolymer comprises a structure of Formula III: 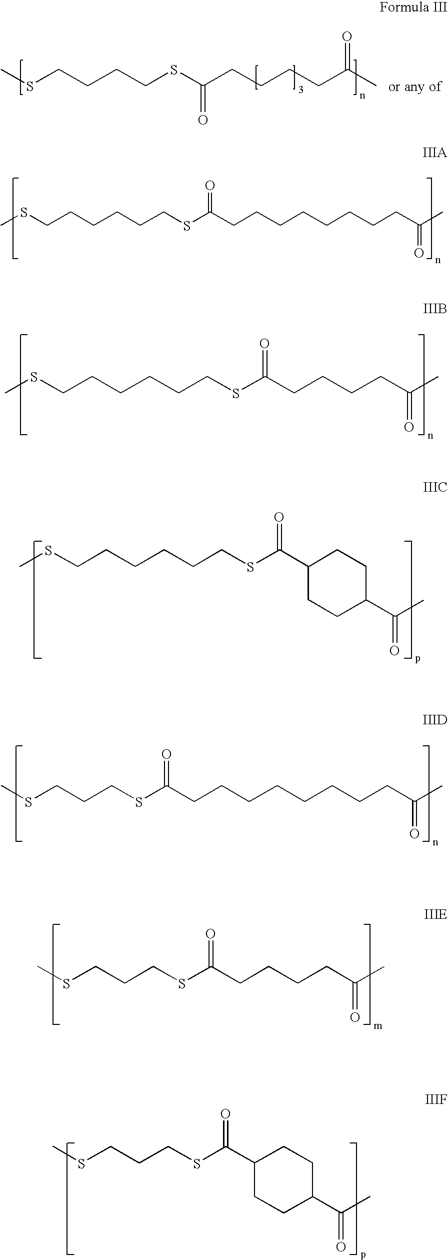
wherein n, m and p are independent positive integers ranging from 1 to about 10,000.

29. The composition of claim 26, wherein the random or block copolymer comprises a structure of Formula I: 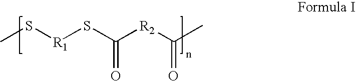
wherein R₁ is an aliphatic group,
wherein R₂ is an organic group, and
wherein n is a positive integer from 1 to about 10,000.

30. The composition of claim 26, wherein the random or block copolymer comprises a structure of Formula III: 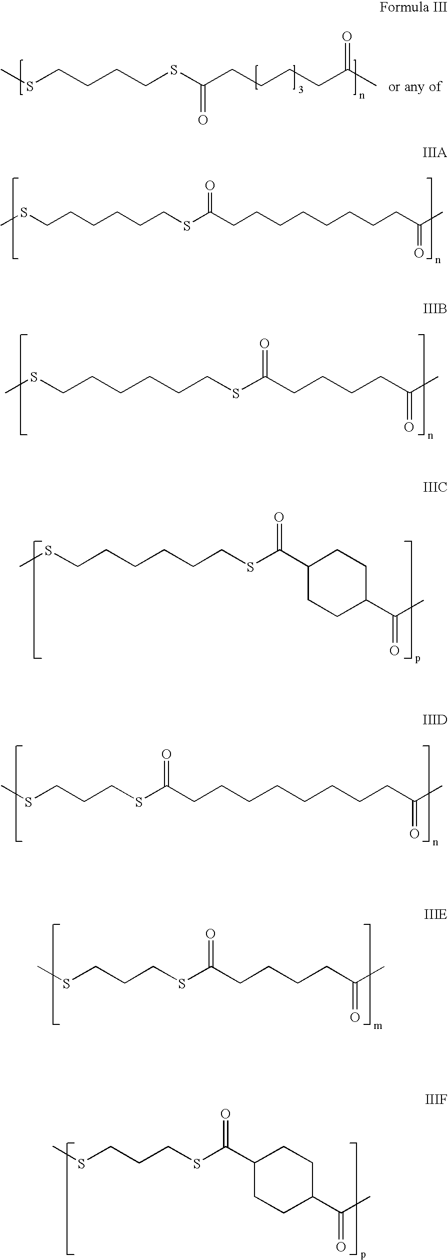
wherein n, m and p are independent positive integers ranging from 1 to about 10,000.

31. The composition of claim 26, wherein the random or block copolymer comprises units derived from a monomer selected from a diol, a diamine, or a combination thereof.

32. The composition of claim 31, wherein the aliphatic di-thiol has a molar ratio (rₙ) from about 0.01 to 0.99,
wherein the diol has a molar ratio (rₘ) from about 0 to about 0.99,
wherein the diamine has a molar ratio (rₖ) from about 0 to about 0.99, and
wherein rₙ+rₘ+rₖ=1.

33. The composition of claim 23, which is formulated into fiber, film, particle or gel formulation.

34. The composition of claim 33, further comprising a bioactive agent.

35. The composition of claim 33, further comprising a bioactive agent selected from paclitaxel, docetaxel, estradiol, 17-beta-estradiol, nitric oxide donors, super oxide dismutases, super oxide dismutase mimics, 4-amino-2,2,6,6-tetramethylpiperidine-1-oxyl (4-amino-TEMPO), tacrolimus, dexamethasone, rapamycin, rapamycin derivatives, 40-O-(2-hydroxy)ethyl-rapamycin (everolimus), 40-O-(3-hydroxy)propyl-rapamycin, 40-O-[2-(2-hydroxy)ethoxy]ethyl-rapamycin, and 40-O-tetrazole-rapamycin, 40-epi-(N1-tetrazolyl)-rapamycin (ABT-578), γ-hiridun, clobetasol, mometasone, pimecrolimus, imatinib mesylate, or midostaurin, or prodrugs, co-drugs, or combinations of these.
